(19) United States
(12) Patent Application Publication
(10) Pub. No.: US 2010/0209943 A1
(43) Pub. Date: Aug. 19, 2010

(54) Measurement Of Protease Activity In Post-Mortem Meat Samples

(76) Inventors: David Goldberg, Boulder, CO (US); Martha Goldberg, Boulder, CO (US)

Correspondence Address: LATHROP & GAGE LLP, 4845 PEARL EAST CIRCLE, SUITE 201, BOULDER, CO, 80301, US

(21) Appl. No.: 12/682,246

(22) PCT Filed: Oct. 9, 2008
(86) PCT No.: PCT/US08/11624

§ 371 Date: Apr. 8, 2010

Related U.S. Application Data
(60) Provisional application No. 60/998,293, filed on Oct. 10, 2007.
(60) Provisional application No. 61/062,711, filed on Jan. 29, 2008.

(51) Int. Cl. G01N 33/573; C12Q 1/37; B01D 57/02

(52) U.S. Cl. 435/7.4; 435/23; 204/450

(57) ABSTRACT

The present methods and systems relate to the placement on meat surfaces of a dye-linked protease substrate bonded in a defined location to a solid, porous support. Proteases in the meat hydrolyze the substrate, which then releases the dye, which diffuses away so that the extent of diffusion can be determined by imaging. Alternatively, the amount of dye released can be determined by its mobilization into a liquid medium. The present methods and systems also relates to determining the amount of hydrolyzed protein that is present in post-mortem meat samples. Agents which bond to hydrolyzed proteins, but not to unhydrolyzed proteins, are generated, wherein such agents comprise antibodies or aptamers. Such agents are then used in quantitative assays comprising ELISA tests or lateral flow tests.

### CROSS REFERENCE TO RELATED PATENT APPLICATIONS

This application is related to and claims priority from Provisional Patent Application No. 60/998,293, filed Oct. 10, 2007, and titled “Measurement of Degradation Enzymes in Post-Mortem Meat Samples”, and from Provisional Patent Application No. 61/062,711, filed Jan. 29, 2008, and titled “Measurement of Protease Degradation Products in Post-Mortem Meat Samples”.

### TECHNICAL FIELD

The present invention relates to the measurement of protease degradation enzymes or products in meat, with particular application to the determination of meat tenderness.

### BACKGROUND

The beef industry would strongly benefit from an objective measure of tenderness that can be used to establish the value of a carcass, and assist cattlemen in breeding better stock. Such a measure will eventually improve the quality of all beef being sold, and increase consumer's satisfaction with beef products. A number of recent consumer tests confirm that there is a strong preference for a tender steak.

Current methods of measuring beef tenderness include a shear test (e.g. the Warner-Bratzler Shear test—WBS), ultrasound measurement, and genetic tests for a variant of the calpastatin gene that correlates with meat tenderness. However, each of these tests suffer from at least one of the problems of high cost, long duration of test, low accuracy and destruction of the meat samples.

Meat becomes much less tender during rigor mortis, after which degradatory enzymes affecting action-myosin, collagen or other proteins then increase tenderness over time. There is evidence that the ultimate tenderness of meat to the consumer is strongly affected by the relative activity of these enzymes. Being able to determine the activity levels of these enzymes would therefore be of great benefit in determining ultimate tenderness.

There are a number of active enzymes, including but not limited to the calpains and the caspases. The enzymes have multiple forms, and furthermore, have inhibitors, agonists, or regulatory factors. For example, the calpains have the calpastatins, which are themselves regulated by the amounts of free calcium. Therefore, to independently understand the activity of a single calpain type, one would need to know the amount of the calpain, the allele of the calpain (different alleles have different activities), the amount of calpastatin, the allele of the calpastatin, and the amounts of free calcium. Furthermore, the activity of these degradatory enzymes are affected by the local pH of intracellular fluid, as well as other environmental factors that may not be understood or identified (e.g. phosphorylation or other chemical modifications that could modulate activity). Furthermore, the activity of these enzymes is also regulated to some extent by autolysis or degradation by other protesases.

Instead, it would be preferable to have a system that measured the protease activity on a “real” substrate, where all of the other factors listed above would be intrinsically taken into account. This could operate either by measuring the activity of proteases in situ within the meat or one the meat surface, or alternatively, by measuring the amount of protease product that is generated in the meat in the early post-mortem period. Such a system would be of substantial benefit to the beef and other meat industries and is the object of the present invention.

### SUMMARY OF THE INVENTION

It would be preferable for the present invention to provide sufficient accuracy of meat tenderness determination in a production environment.

It would also be preferable for the present invention to provide inexpensive meat tenderness determination in a production environment.

It would further be preferable for the present invention to provide non-destructive meat tenderness determination in a production environment.

It would additionally be preferable for the present invention to determine meat tenderness using an automated system in a production environment.

It would yet further be preferable for the present invention to provide a determination of meat tenderness that is rapid and can be performed on a meat processing line between the time that the rib-eye used in grading is exposed and the disposition of the carcass is determined subsequent to USDA grading.

Additional objects, advantages and novel features of this invention shall be set forth in part in the description that follows, and will become apparent to those skilled in the art upon examination of the following specification or may be learned through the practice of the invention. The objects and advantages of the invention may be realized and attained by means of the instrumentalities, combinations, and methods particularly pointed out in the appended claims.

In accordance with the purposes of the present invention, as embodied and broadly described herein, the present invention is generally directed to a method for determining tenderness of a meat sample. A test strip can be placed on the meat sample, wherein the test strip comprises a solid support and a protease substrate bound at a discrete location on the support. The protease substrate can be labeled with an optically-detectable marker, and the protease substrate and its marker can be inhibited from diffusing from the discrete location. The test strip can then be put into contact with fluids from the meat sample, so that the fluids bathe the labeled protease substrate. The test strip can then be incubated for a predetermined period of time, in which proteolysis hydrolyzes the protein substrate and releases the marker from the discrete location. The diffusion of the optically-detectable marker from the discrete location can then be measured. Then, the tenderness of the meat sample can be determined using an automated decision algorithm that uses the amount of diffusion of the marker.

The protease substrate can comprise a structural protein, a polypeptide comprising multiple protease recognition sites, or a mixture of a multiplicity of polypeptides. The predetermined period of time can be less than 20 minutes, and the incubating can be performed at a temperature greater than 30° C.

The measuring means can comprise an imager or a scanner, and can occur while the test strip is in contact with the meat sample, or while the test strip is not in contact with the meat sample. The measuring can further comprise measuring the optically-detectable marker at the discrete location, or obtain an indication of the pH of the meat sample from an indicator on the test strip, or obtaining an indication of the concentration of calcium ions within the meat sample from an indicator on the test strip.

The present invention can also be directed to a system for determining tenderness of a meat sample that comprises a solid support, a protease substrate attached to the support in a discrete location, a marker that is attached to the protease substrate, wherein the marker can be measured by optical means, a surface of the support that can be exposed to the meat sample and which is absorbent of fluids from the meat sample, thereby bathing the support and its attached protease substrate in those fluids, an optical profiler that optically profiles the support, wherein the profile provide indication of the spatial distribution of the marker; and a computer that analyzes the images to determine the degree of diffusion of the marker from the discrete location, and which determines the tenderness of the meat sample using the degree of diffusion.

The marker can be visible or fluorescent. The protease substrate can comprise a polypeptide that contains a multiplicity of protease recognition sequences.

The system can further comprise an incubator for maintaining the temperature of the solid support while the support and its attached protease substrate are bathed in the fluids from the meat sample.

The present invention can additionally be directed to a method for determining tenderness of a meat sample that comprises obtaining a protein sample from the meat sample comprising a protein breakdown product resulting from post-mortem proteolysis and a reference, measuring at a predetermined time post-mortem from the protein sample the amount of the protein breakdown product relative to the reference; and determining tenderness of the meat sample using an automated decision algorithm that uses the ratio of the amount of the protein breakdown product to the amount of the reference.

The reference can comprise the source for the protein breakdown product, or it can comprise a protein that is not the source for the protein breakdown product.

The step of measuring can further comprise optical profiling.

The method can further comprise physically separating the protein breakdown product and the reference by differences in their physical characteristics or by differences in their binding to a specific binding agent.

The present invention can yet also be directed to a system for determining tenderness of a meat sample that can comprise a sample collector, for obtaining a protein sample from the meat sample comprising a protein breakdown product and a reference; a protein quantifier, for measuring the quantity of protein breakdown product relative to the reference; and a computer, for determining tenderness of the meat sample using an automated decision algorithm that uses the ratio of the quantity of the protein breakdown product to the quantity of the reference as measured by the protein quantifier.

The system can further comprise a protein separator for separating the protein breakdown product and the reference. The protein separator can separate the protein breakdown product and the reference by differences in their binding to a specific binding agent, or by differences in their physical characteristics. The protein separator can comprise a solid support, wherein subsequent to separation, the protein breakdown product is at a first location on the solid support, and the reference is at a second location. Also, the protein separator can comprise a throughput device, through which the protein breakdown product and the reference pass at different times. The protein separator can comprise the sample collector.

The sample collector can be covered in part with a non-porous coating comprising a gap in the coating at a discrete location, so that placement of the sample collector on the meat sample allows the protein sample to be collected at only that discrete location.

The present invention can yet additionally be directed to a method for determining tenderness of a meat sample, which can comprise obtaining a protein sample from the meat sample, wherein the protein sample comprises a protein breakdown product resulting from post-mortem proteolysis and a reference; separating the protein breakdown product from the reference on the basis of their physical characteristics; measuring the quantity of the protein breakdown product relative to the reference; and determining tenderness of the meat sample using an automated decision algorithm that uses the measured relative quantity.

The separating can comprise ion-exchange, chromatography, electrophoresis or mass spectroscopy, which can be preceded by laser desorption.

The obtaining can comprise contacting the surface of the meat sample with a separation medium.

The present invention can yet further be directed to a system for determining tenderness of a meat sample, which can comprise a separation medium, in which a protein sample from the meat sample is collected, wherein the protein sample comprises a protein breakdown product resulting from post-mortem proteolysis and a reference; a separation impetus that separates the protein breakdown product from the reference within the separation medium; a protein quantifier that measures the relative quantities of the separated protein breakdown product and the reference; and a determiner that determines tenderness of the meat sample using an automated decision algorithm that uses the measured relative quantities as measured by the protein quantifier.

The present invention can alternatively be directed to a method for determining tenderness of a meat sample, which can comprise obtaining a protein sample from the meat sample, wherein the protein samples comprises a protein breakdown product resulting from post-mortem proteolysis of a source protein, a protein breakdown complement that is formed coincidentally to the protein breakdown product during formation by proteolysis of the source protein, and a reference; separating the protein breakdown product from the source protein and the protein breakdown complement using a binding reagent that binds to the protein breakdown product and which does not bind to the source protein, the protein breakdown complement or the reference; measuring the quantity of the protein breakdown product relative to the reference; and determining tenderness of the meat sample using an automated decision algorithm that uses the measured relative quantity.

The binding reagent can be selected from the group consisting of antibodies, antibody fragments, aptamers, lectins and membrane receptors.

The separating can further comprise attaching the binding reagent to a location on a solid support, and collecting the protein breakdown product at that location, or alternatively binding a second binding reagent to the reference, which is attached to the solid support at a second location on the solid support.

During the separating, the protein breakdown product and the reference can contact first the first location, or the protein breakdown product and the reference contact first the second location.

The reference can comprise the source protein.

The present invention can yet alternatively be directed to a method for determining tenderness of a meat sample, which can comprise a specific binding agent that binds to a protein breakdown product formed by proteolysis of a source protein post-mortem in the meat sample, and which does not bind to the source protein, a protein breakdown complement, or a reference; a test strip on which the amount of the protein breakdown product is measured; an optically-detectable marker that marks the protein breakdown product; an affinity linker associated with the optically-detectable marker that links the marker to the protein breakdown product; an immobilizer that immobilizes the protein breakdown product to a location on the test strip; an optical profiler that captures a profile of the optically-detectable marker on the test strip; and a computer that computes the quantity and spatial distribution of the optically-detectable marker from the profile, and that determines the tenderness of the meat sample from that computed quantity and spatial distribution.

The system can further comprising a second immobilizer, immobilizing the reference product to a second location on the test strip, wherein the optically-detectable marker additionally marks the reference.

The present invention can yet also further be directed to a method for determining tenderness of a meat sample, which can comprise transferring a protein sample from the meat sample onto a starting location on a test strip; transporting the protein sample from the starting location to a labeling location; labeling a protein breakdown product within the protein sample with an optically-detectable marker at the labeling location; conveying the labeled protein breakdown product to a binding location; binding the labeled protein breakdown product to the binding location; profiling the optically-detectable marker at the binding location in order to determine the quantity of protein breakdown product in the protein sample; and determining the tenderness of the meat sample from the quantity of the protein breakdown product.

The step of labeling can further comprise binding to the protein breakdown product a labeled binding agent that is specific for the protein breakdown product and that is stored at the labeling location to the protein breakdown product.

Binding can comprise binding the labeled protein breakdown product to a binding agent that is specific for the protein breakdown product and that is attached to the test strip at the binding location.

The test strip can comprise a lateral flow test strip.

The present invention can yet also further alternatively be directed to a method for determining tenderness of a meat sample, which can comprise transferring a protein sample from the meat sample onto a starting location on a test strip, wherein the protein sample comprises a protein breakdown product and a reference; conveying the protein breakdown product and the reference along the test strip, wherein the movement of the protein breakdown product and the reference are at different rates; staining the test strip so as to make the protein breakdown product and the reference optically-detectable; profiling the test strip with an optical profiler so as to measure the quantities of protein breakdown product and the reference, which are at distinguishable locations on the test strip; and determining the tenderness of the meat from the quantities profiled by the optical profiler.

The test strip can comprise a chromatography strip.

Transferring can further comprise contacting the test strip to the meat sample. The surface of the test strip contacting the meat sample can be coated with a non-porous coating comprising a gap, wherein transfer of the protein sample to the test strip is limited to the location of the gap.

### BRIEF DESCRIPTION OF THE DRAWINGS

FIG. 1A is a schematic of a labeled analase substrate attached to a support.

FIG. 1B is a schematic that shows the labeled analase substrate of FIG. 1A after it has been cleaved by the analase.

FIG. 2A is a side cross-section schematic of test strip with the labeled analase substrate on a solid support initially placed on a meat sample.

FIG. 2B is the test strip of FIG. 2A after the test strip has wicked up fluid from the meat sample.

FIG. 2C is the test strip of FIG. 2B after analase has cleaved analase substrate on the test strip, allowing diffusion of the label away from the bound analase substrate.

FIG. 3 is a top view schematic of a test strip 400 with four different spots 300, each with a different analase substrate 150, and including a topological calibration mark.

FIG. 4 is a schematic diagram of a reader 700 for reading test strips 400.

FIGS. 5A and 5B are compound figures of chromatography of a meat sample, comprising a schematic cross-sectional view of the chromatogram, and a graphical view of absorbance.

FIG. 6A is a schematic diagram of a protein A that is degraded in situ by proteases to form degradation products B and C, in which product B has a Selective Binding Agent.

FIG. 6B is a schematic diagram of a lateral flow test for detection of the protease degradation product B, which product is described in FIG. 6A.

FIG. 7A is a schematic diagram of a protein A that is degraded in situ by proteases to form degradation products B and C, in which products B and C have Selective Binding Agents.

FIG. 7B is a schematic diagram of a lateral flow test for detection of the protease degradation product B, which product is described in FIG. 7A.

FIG. 8 is a side schematic view of a chromatographic separation strip.

### DESCRIPTION OF THE INVENTION

### Overview

It is known that calpain protease activity is related to the tenderness of aged meat. The activity of calpain proteases, however, is known to vary between Bos taurus and Bos indicus, the different isoforms of calpain (e.g. mu- and m-), the allele of each calpain isoform, the concentration and allele of the inhibitor calpastatin, the pH, the calcium concentration, and other factors. Even if one were able to measure the concentration of calpain or calpastatin with some accuracy (and such methods have not to date been either accurate, inexpensive, non-destructive or rapid enough for commercial use), given the large number of other factors that affect calpain activity, it is likely that such a tool would not be highly predictive of tenderness. Furthermore, it is known that there are a large number of other proteases present in post-mortem meat samples that may contribute to the aging process (e.g. the caspases and cathepsins), and given that each of these proteases are likely to be subject to the same variety of variations and modulators as calpains, it is not clear how easy it will be to develop a methodology based on the measurement of the concentrations of specific proteases and their component systems.

Instead, there are generally two classes of methods that can be used to determine protease activity in post-mortem samples. In a first method, meat samples are provided to protease substrates that are treated, such as by dye labeling, in such a way as to allow the degree of proteolysis to be monitored. In a second method, one or more products of in situ proteolysis are measured quantitatively, along with the time or measurement, to give an indication of the amount and rate of proteolysis within the meat sample.

### Measuring Protease Activity

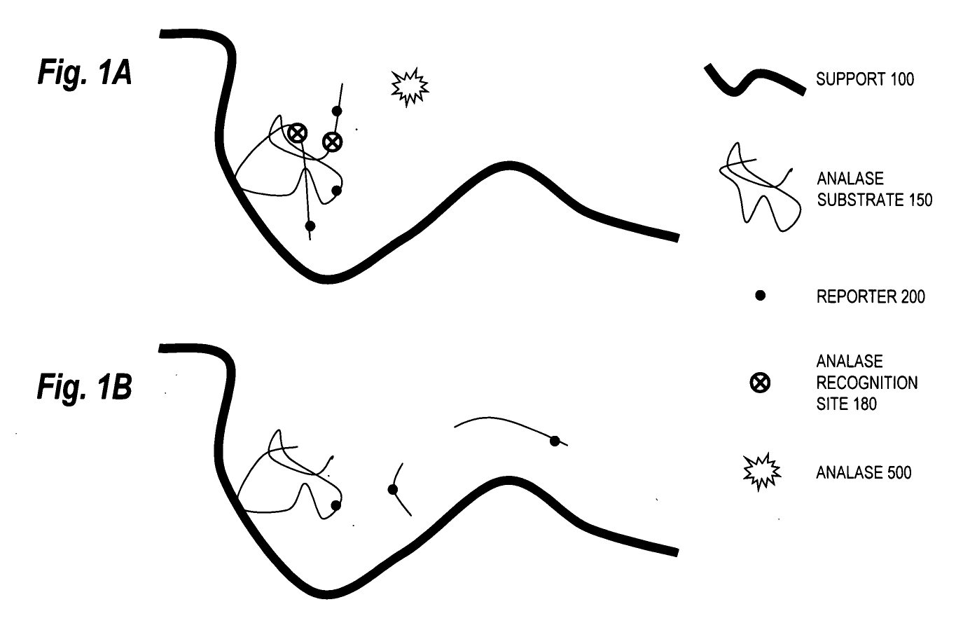

FIG. 1A is a schematic of a labeled analase substrate 150 attached to a support 100 via a linker 110.

The analyte being measured is called an analase 500, as the analyte has enzymatic degradatory activity against a substrate (the analase substrate 150). The analase substrate 150 is generally a protein such as actin, myosin, desmin, collagen, elastin, proteoglycans, or other generally structural proteins present in large quantities within muscle tissue. Another analase substrate 150 can comprise synthetic substrates that comprise the target amino acid sequences for enzymes that are present in post-mortem beef samples. Such target amino acid sequences comprise leu-leu-val-tyr for m- and mu-calpain, asp-glu-val-asp for caspases 3, 6, 7, 8 and 10, tyr-val-ala-asp- for caspases 1 and 4, and leu-glu-his-asp for caspases 4, 5 and 9. For example, a substrate with the sequence (leu-leu-val-tyr)_{N}—where N is from 2 to 20 can be used in the detection of m- and mu-calpains. The target amino acid sequences listed above are simple examples of such target sequences, and the actual target sequences for the proteases listed above and of interest in such a study comprise a variety of factors, many of which are poorly understood, and which often involve amino acid sequences far longer than those given above. Thus, it is within the spirit of the present invention to replace the target sequences above with other such amino acids sequences that may be determined in the future are more specific or of higher reactivity to the proteases, and that multimers of such target sequences are also of benefit in such an assay.

It should also be noted that the enzymatic activity can also be directed against other structural or other elements in tissue, such as DNA or protein cross-linking moieties.

The analase substrates 150 can be purified or semi-purified compounds, which can be extracted from beef or swine tissue, or generated from single-cell sources (bacteria or yeast) or other sources (plants, milk, etc.) where organisms are produced that yield large amounts of the desired substrate. These proteins can be treated in such as way as to retain their natural secondary, tertiary and quaternary structure, or alternatively, may be denatured by heating, precipitation, etc or even partially degraded by proteases.

Alternatively, heterogeneous preparations from tissue can be used instead. For example, whole beef muscle can be ground, the fat removed by solvent extraction, and then the remaining portion can be concentrated without denaturation, or alternatively precipitated with alcohol or other precipitation means. If whole samples are to be used, it is useful to denature the sample by heat and/or solvent precipitation so that any intrinsic degradatory enzymes are inactive and not continuing to act on the sample.

It is generally preferable for the substrate not to be highly coagulated, so that it is largely soluble or in suspension in very small particles (preferably less than 50 microns, and more preferably less than 10 microns, and most preferably less than 5 microns). Thus, if making precipitates, it is preferable to perform the precipitation in a dilute suspension. Alternatively, larger coagulates can be treated with a protease (e.g. trypsin) with an amino acid sequence specificity different from that of the proteases under investigation. Once the sample is solubilized, the protease activity can be extinguished by heating, the use of covalent or non-covalent inhibitors (e.g. PMSF), or other inhibitory means.

The analase substrate 150 is then labeled with an optically-detectable reporter 200, which is conveniently either a chromophore or a fluorophore. If light absorbing, it is convenient if the wavelengths absorbed are in the visible spectrum, but any spectrum for which stand-off imagers or colorimeters or spectrophotometers could determine the spatial distribution of the label on a two-dimensional field would suffice. Examples of fluorophores include derivatives of fluorescein, rhodamine, coumarine, cyanine, Alexa fluors, and DyLight fluors. Examples of chromaphores include a wide range of azo compounds, lycopenes, anthocyanins, beta-carotenes, phytochromes, porphyrins, and bilirubins. An alternative reporter 200 would comprise luciferin, which when released would be available for luminescence in the presence of luciferase. A further alternative would include Green Fluorescent Protein, or other proteins that show fluorescent or luminescent behavior (e.g. luciferase).

Yet another alternative is to use a Förster resonance energy transfer (FRET) system, comprising a donor chromophore and an acceptor chromophore that are within approximately 10 nm of one another in the initial, associated state. In such a state, the fluorescence occurs preferentially at the fluorescence emission wavelength of the acceptor chromophore. The connection between the donor and acceptor chromophore is a target polypeptide that is susceptible by cleavage by the protease under investigation. As the target polypeptide is cleaved, the donor and acceptor polypeptide are dissociated, and the fluorescence then occurs preferentially at the emission wavelength of the donor chromophore. A number of such paired donor chromophores (e.g. cyan fluorescent protein or EDANS) and acceptor chromophores (e.g. yellow fluorescent protein, DABCYL, DABSYL) are known in the art. In such case, the donor or the acceptor chromophores can be affixed to the support 100, and upon protease action, there would be increase fluorescence that occurs not only outside of location of the bound chromophore on the substrate 100, but also within the location. This provides additional quantitative information on the amount of protease.

It is highly preferable for the reporter 200 to be Generally Regarded As Safe (GRAS), to be approved as a food additive, or to be able to be approved as a food additive, as the reporter 200 can become in contact with meat that will be for sale. It should be appreciated, however, that the total amounts that will get into contact with the meat will be very small (certainly no more than picogram or nanogram amounts), and that there may be means to limit its contact with meat (see below).

The reporter 200 is chemically reacted with the analase substrate 150 through, for example, reaction with primary amines, sulfhydryls, carboxyls, or oxidized carbohydrate groups (e.g. through aldehydes) on the protein. The use of such linking reagents is well known in the prior art.

The reporter 200 labeled analase substrate 150 is then attached to the solid support 100, preferably in the form of a thin, porous paper-like format, wherein the support is preferably hydrophilic, and wherein the support aggressively wicks aqueous fluid up contact by capillary action. An example of such support material is cellulose or polystyrene, and may also include biodegradable materials, such as polylactide, polyglycolide, polycaprolactone, polyhydroxybutyrate, dextrans, starches, such that any materials that are not removed in the process will, over time, degrade and thereby remaining material will have less impact on the salability of the meat. The linkage between the labeled analase substrate 150 and the support 100 can utilize a variety of chemical reactions similar to those used for reacting the reporter 200 to the analase substrate 150. This linkage can employ linkage molecules with two or more reactive sites (one for attachment to the support 100 and one for attachment to the analase substrate 150), or alternatively, the support 100 can come with chemical moieties along its backbone for attachment.

It should be noted that the notion of an analase substrate 150 is that there are analase recognition sites 180 in the analase substrate 150 such that the addition of analase 500 would cleave the analase substrate 150 at or near the recognition sites 180.

FIG. 1B is a schematic that shows the labeled analase substrate 150 of FIG. 1A after it has been cleaved by the analase 500, such as would occur after the addition of post-mortem meat samples containing the analase 500. The analase substrate 150 is cleaved multiple times by the analase 500, releasing some of the reporter from attachment to the support 100, such that the reporter 200, attached to freed fragments of the analase substrate 150, is now soluble and can diffuse away from the point of attachment of the analase substrate 150.

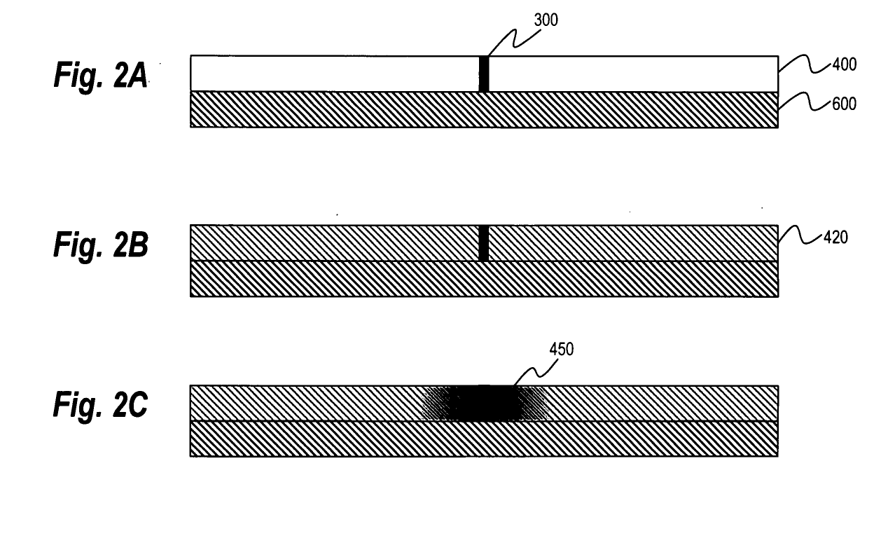

FIG. 2A is a side cross-section schematic of a test strip 400 with the labeled analase substrate 150 on a solid support 100 initially placed on a meat sample 600. The labeled analase substrate 150 is located in a limited region, which is shown here as a spot 300 of labeled substrate 150 that extends through the thickness of the test strip 400. It should be noted that the spot does not need to be of constant breadth throughout the thickness of the test strip 400, for example if the labeled analase substrate 150 is spotted on top of the test strip 400 during manufacture, in which case the diameter of the substrate 150 on the top of the strip 400 will generally be larger than at the bottom of the strip 400. The meat sample 600 is preferably, but not necessarily, the surface of a cross-sectional cut between the 12^{th} and 13^{th} ribs of a beef carcass, which is normally exposed during the process of USDA grading.

FIG. 2B is the test strip of FIG. 2A after the test strip 400 has wicked up intracellular fluid from the meat sample 600. The saturated test strip 420 will contain an amount of analase 500 that in some manner corresponds to the amount of analase 500 present in the meat sample 600.

FIG. 2C is the saturated test strip 420 of FIG. 2B after analase 500 has cleaved analase substrate 150, allowing diffusion of the reporter 200 away from the bound analase substrate 150, creating a colored area 450 laterally from the spot 300. The diffusing reporter 200 corresponds to the situation of FIG. 1B, where some reporter 200 is no longer bound to the attachment site on the support 100. For a given time after the introduction of analase 500 onto the test strip 400, the radius and/or the intensity of the colored area 450 is then indicative of the amount of analase 500 in the meat sample 600. The size and/or intensity of the colored area 450 can be measured with an imager, which can be a CCD or CMOS imager, and which can also have fluorescent illuminators and filters should the reporter 200 be a fluorophore rather than a chromophore.

The imager should generally be high resolution, inasmuch as the spot 300 diameter will preferably be less than 1 mm, and more preferably less than 500 microns, and most preferably less than 200 microns, and the diameter of the colored area 450 spread from the location of the original spot will generally be in hundreds of microns. The resolution of the imager will preferably be less than 100 micron, and more preferably less than 50 micron, and most preferably less than 25 microns. If a high-resolution imager is being used to examine meat fiber characteristics, for example, to correlate with tenderness, the same imager can also conveniently be used to measure colored area 450 characteristics, as well.

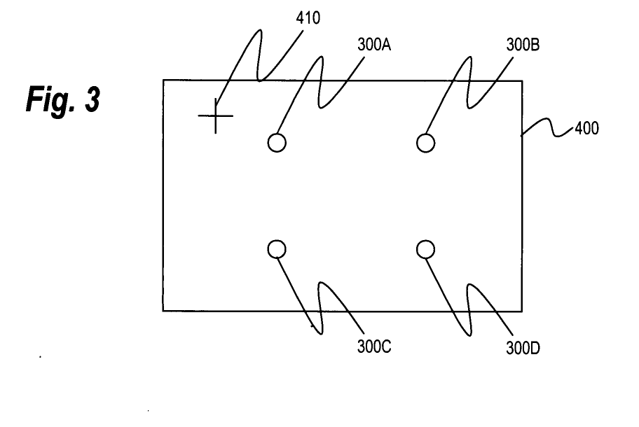

FIG. 3 is a top view schematic of a test strip 400 with four different spots 300, each with a different analase substrate 150. For example, the spot 300A can have a purified actin substrate 150, while the spot 300B can have a purified collagen substrate 150, whereas the spot 300C can have a total soluble meat protein extract substrate 150, whereas the spot 300D can have an insoluble meat protein extract that is solubilized by protease substrate 150. The size and intensity of the areas around all four spots 300A-D would be indicative of a broad range of analase activities (e.g. collagenases, calpains, caspases, etc.). One or more calibration spots 410, which may be cross-hair, bulls-eyes, or other shape, can be used to determine which spot 300 is which. Additionally, the reporters 200 associated with each spot can be different in characteristic (e.g. color, or fluorescence emission frequency), so as to unambiguously define which spot 300 is which. In addition, color calibration can be included onto the test strip 400, so as to determine the color balance of the illuminator, as well as to account for the color of the intracellular fluids that are absorbed by the test strip 400, and which may also include small deposits of blood that may, from time to time, be present in the meat. Given that the intracellular fluids and blood have a hue that is very close to a pure red, it is preferable for the reporter 200 to have strong blue or green color elements, that will be easy to distinguish from any intracellular fluids or blood.

The image that is obtained from the imager will be examined by a computer program to determine the extent and intensity of the colored area 450. While this information can give a specific activity of amylases or proteases or other degradatory enzymes, in general, it is preferable for this information to be used in the generation of algorithms that correlate (in broad terms) the degree of spreading with meat tenderness or other meat characteristics. Such algorithms might include statistical correlations, linear regressions, nonlinear regressions, binning, tree analysis, Bayesian statistics, neural networks, support vector machines, and more.

It is preferable for the strip, after being in contact with the meat sample, and having absorbed intracellular fluids, to be removed from the meat sample. The reason for this is that there is generally only a short period of time between when the meat surface is exposed (e.g. a cut between the 12^{th} and 13^{th} ribs) and the when the result is needed within the commercial operation for use in determining how the carcass is to be handled. This time is frequently only 8-10 minutes in a commercial meat packing plant. In general, it is preferable for the time of incubation of the meat sample with the test strip to be less than 20 minutes.

Due to the short time, it is preferred that the sample be warmed so as to increase the rate of proteolysis, with an optimal temperature above 30° C. For example, in general, enzyme rates double for every 10 degrees Centigrade, so a sample held at 37° C. will have approximately 10 times the proteolysis of a similar sample held at 4° C.

### Additional Indicators

It should also be appreciated that other indicators can be placed on the test strip 400 to provide other information that can assist in the determination of protease activity. That is, there are many factors within the meat that affect protease activity, and chief among these appears to be the intracellular pH. Thus, it is convenient to have a colorimetric pH indicator on the strip, wherein it is preferable for the range over which the indicator to be functional to include pH from 5.5 to 6.5, and more preferable for the range to include from 5.5 to 7, and most preferable for the range to include from 5.0 to 7.5. Examples of such indicators include anthocyanins, hematochromes, flavenoids, azolitmins, orceins, and triphenylmethanes.

In addition, calcium chromophores can also be included on the test strip 400, so as to provide quantitative indication of the levels of free calcium in intracellular fluids. Such calcium chromophores comprise calcein, eriochromes, murexide, hydroxynapthol blue, methylthymol blue, pyrogallol, chromazurol, calmagite, and other compounds and their derivatives.

### Strip Reader

It should be noted that the dye diffusion can be monitored on the strip 400 while it is located on the carcass being measured, but it is alternatively within the spirit of the present invention for a reader 700 to be provided, into which the strip 400 can be placed for incubation of the proteolysis and/or reading of the dye diffusion. The use of the reader 700 has the advantages that it can provide more rapid proteolysis by maintaining an elevated temperature, that it can provide more consistent illumination during reading (which is especially useful for fluorescence imaging), it can provide more consistent distance between the imager and the sample, and that it requires less precision in placement on the imager, which can be difficult on the carcass since the carcass is in continuous movement on the grading line.

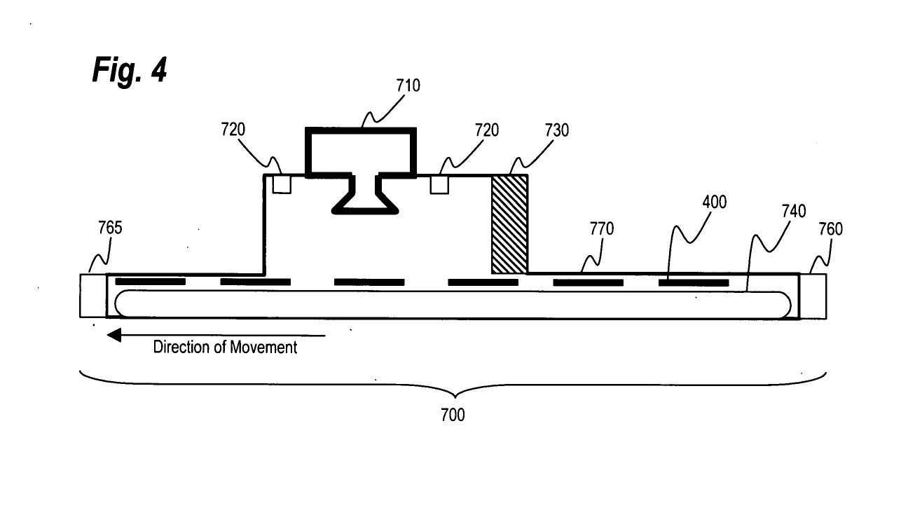

FIG. 4 is a schematic diagram of a reader 700 for reading test strips 400. The reader 700 is surrounded by a light-tight enclosure 770. A heater 730 provides a regulated temperature environment, which is preferably around 37° C. Internal illumination is provided by one or more illuminators 720, which can be LEDs, fluorescent lamps, lasers, arc lamps or other illuminators, which can be supplemented with diffusers (not shown) to provide uniform illumination.

Test strips 400 are introduced into an entry port 760, which can be light-tight or simply a hinged entry in which the length of a constricted aspect of the enclosure 770 (i.e. with little clearance) does not allow much ambient illumination into the central chamber of the reader 700. The strips 400 are transported with an automated feed 740 into the central chamber of the reader 700. This transport can be rapid (i.e. over a period of seconds) should the strips 400 be incubated for proteolysis in a different location or on the carcass, or can alternatively take a predetermined amount of time to allow for proteolysis to occur on the strip (e.g. 5-20 minutes).

When the test strip 400 is in position under an imager 710, which can be a CCD or CMOS imager, or which can alternatively be a scanning laser imager, an image of the strip 400 is obtained. This image can be transferred to a computer for image analysis (not shown). The strip 400 is then transported to an exit port 765 for removal, or alternatively can be transported to a waste store where the strips 400 are collected for later disposal.

### Proteolysis Measurement Directly on the Carcass

The methods above have the difficulties that the test strip 400 must be removed from the meat on which it is placed prior to processing or sale, and that the large volume of fluid that the strip 400 can hold might require time for completely and uniformly saturating the strip 400 (and there is generally only a limited amount of time, from perhaps 8 to 15 minutes over which the test can be performed if it is to be performed on the meat grading line of a conventional meat processing facility). A number of methods below can be used to overcome some or all of these concerns.

As mentioned above, if the test strip 400 is made of a labile support 100, then the strip 400 might be able to be left on the meat, and the strip 400 could then be allowed to decompose and diffuse away. For example, there will generally be enough amylase in a sample such that a starchy strip 400 would, over a period of time, decompose into products that would generally have little effect on the quality of the meat. Other materials, such as polylactides, polyglycolides, polycaprolactones, and polyhydroxybutyrates may degrade on their own, without the requirement for specific enzyme assistance.

Alternatively, the support can be fashioned into microspheres, with diameters preferably between 20 and 100 microns, and more preferably between 10 and 200 microns, with a variance of less than 10% in size. These spheres will be made preferably of degradable material, as above, though their very small volume will generally not affect meat palatability. The spheres will generally be hydrophilic, to the extent that intracellular fluid will be drawn into spheres that are contacting the meat via capillary action. In the case of such sphere, two different methods could be used to detect the presence of analase.

In a first method, reporter 200 that is released will diffuse into the adjacent meat in similar fashion that the intracellular fluid was drawn into the bead. The amount of diffusion will be roughly proportional to the surface area contact between the meat and the sphere, and this will vary potentially strongly from sphere to sphere. To overcome this natural variation, the spread of reporter 200 from a large number of different spheres can be measured. It is assumed that the spheres will be deposited via an airsteam that carries many of the spheres, so that thousands of the spheres can be monitored over a small area of meat.

In a second method, the spheres will have a core of labeled analase substrate 150, which will be encapsulated in some amount of material that is lacking the reporter 200. For example, a core of 20 micron diameter material with large amounts of labeled analase substrate 150 can be surrounded by material lacking labeled analase substrate, making a sphere of approximately 50-200 microns. The reporter 200 does not need to diffuse into the meat, but rather the color and/or intensity of the entire sphere can be determined.

In the cases above, in order to determine the activity of multiple analases 500, spheres of different colored material, or with different colors of reporter 200, or of different diameter can be used.

In a yet different alternative methodology, spots 300 can be “printed” onto meat with processes and equipment very similar to that of ink jet printers. In such cases, the methodology employed can include thermal ink jets, piezoelectric methods, and continuous ink jet methodologies. Because the labeled analase substrate 150 is printed directly onto the meat, the diffusion of analase 500 to the analase substrate 150 is not necessary. Because the printing will in general result in spots of particular topological relationships, allowing the spots corresponding to each analase substrate to be distinguished by position, a single reporter 200 can be used. Because the volumes of each spot are very small (measured in picoliters), the amounts of contamination of the meat can be miniscule. In general, ink jet technologies are extremely consistent, so that the variations in drop size will be small.

One benefit of ink jet technologies is that they are non-contact, whereas many other printing technologies are contact. One concern with a contact technology is that analase 500 from one meat sample will be picked up onto the printer plate and then degrade analase substrate 150, possibly in the larger reservoir holding bulk substrate 150. Also, it can be that contact printers may transfer bacterial contaminants from one meat sample to the next. These concerns, it should be appreciated, can be overcome with methods including the use of denaturing solvents and heat (possibly in concert with one another). For example, a printing platen can be kept at a higher temperature to kill bacterial and denature contaminating analase 150 (though small enough not to affect bulk meat temperature), and also be sprayed with ethanol between samples for similar effect, wherein the heat quickly vaporizes the ethanol so that it is not transferred onto the meat itself.

### Measurement of Protease Products

Instead of measuring protease activity, the product of protease activity in meat can alternatively be performed. Such protein degradation products are present in large abundance 12-36 hours after slaughter. To achieve this goal, one needs to identify both a suitable marker for proteolysis, as well as a commercially acceptable assay platform.

Levels of proteolytic activity are highest in the first day or so post-mortem, and then decrease over time. The aging process for meat occurs over 14-28 days, and while the amount of degradation increases, the levels of proteolytic enzymes decrease (through autolytic activity). If we approximately integrate the protease activity curve over the first 21 days, the fraction of proteolysis that is expected to take place in the first 2 days is on the order of 15-30% of the total proteolytic degradation.

Consider a protein A that has been hydrolyzed, which has two degradation products B and C. There are a number of methods that can be used to detect the hydrolysis.

### Separation by Physical Characteristics

In a first case, we can separate the mixture by physical characteristics, such as molecular, charge, hydrophilicity, and more, as can be accomplished by column, paper or gel chromatography, or by gel electrophoresis. The species A, B, and C will be represented by individual peaks in the eluate or the completed chromatogram or gel. The more the proteolytic degradation, the more B and C that will be present relative to A. If the separation is by a gel, column or other means, the relative degradation of many proteins A1, A2, etc. into products B1, C1, and B2, C2, etc can be monitored.

In practice, after the ribeye has been exposed, a small sample of intracellular fluid can be obtained by capillary action, either with a pulled glass pipette or a small diameter tube or by placing a portion of paper substrate (e.g. filter paper) onto the meat. If collected with a glass pipette or small diameter tube, the contents can then be transferred to a filter paper by contact. The location of the meat fluid is called the “spot”.

One method of applying the meat fluid is to have a backing to the test strip that is impermeable to fluids, but which is absent in a small portion beneath the spot. Placing the strip on the meat will allow only the spot to wick up fluid.

One end of the paper is then placed into a transport fluid reservoir (i.e. the fluid that will carry the proteins along the test strip), or a portion of transport fluid is placed on the paper on one side of the spot, and the contents of the meat fluid are separated by physical characteristic. Instead of paper, a packed material (e.g. silica gel or alumina and chemical modified forms thereof) on a solid substrate can alternatively be used. The paper or material can be impregnated with positive charges, negative charges, or hydrophobic materials, and the transport fluid can contain detergents, salts, acids, bases, buffers or organic solvents to aid in the separation, which may occur via a number of mechanisms, including adsorption chromatography or ion exchange chromatography. Instead of paper, a beaded, gel or packed material can be used to allow for molecular exclusion chromatography or other means that separate according to size (e.g. ion exchange in the presence of ionic surfactants).

At the conclusion of the separation, a dye, stain or other means is used to indicate the presence and location of proteins. Such stains can include bromocresol green, amido black 10B (Napthol Blue Black), ninhydrin, and o-tolidine.

It should be noted that paper or planar chromatography is most useful for very low molecular weight species, and that the methods above can be similarly used with electrophoresis instead of fluid flow on paper, silica gel, etc. For example, the test system can be set up with acrylamide or starch or similar gel electrophoresis. In order to stain for proteins, gel stains such as Coomassie stains can be used, as well as fluorescent stains, but it should be appreciated that the gel preferably be very thin to allow rapid staining and clearing of the gel.

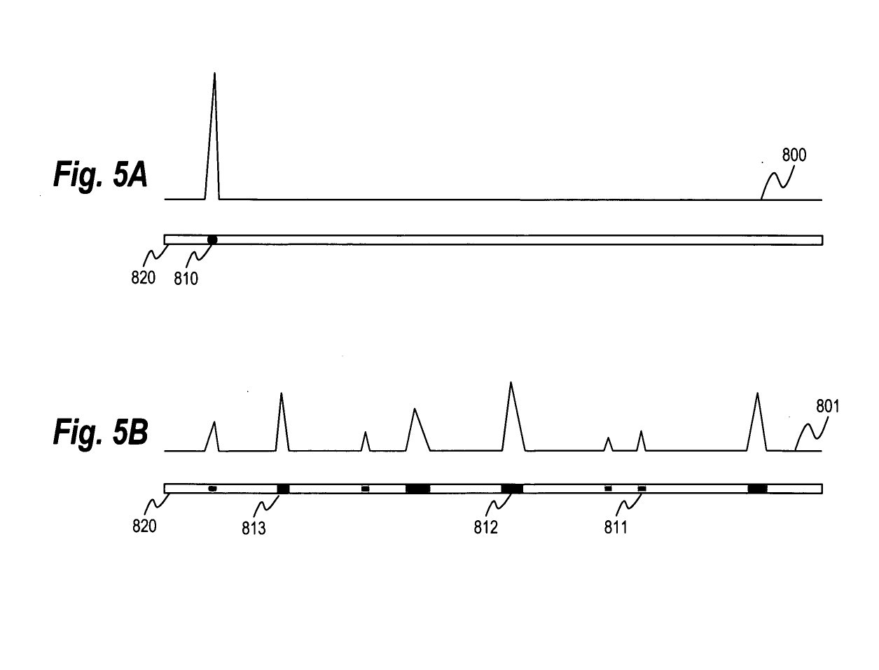

FIGS. 5A and 5B are compound figures of chromatography of a meat sample, comprising a schematic view of the chromatogram, and a graphical view of absorbance. In FIG. 5A, chromatogram 820 comprises a spot 810 on which a meat sample has been placed. A graph 800 shows the distribution of protein on the chromatogram 820, if the chromatogram were to be stained to show protein locations. At the location of the spot 810, a large absorbance peak is shown.

In FIG. 5B, the chromatogram 820 has been developed so as to separate proteins on the basis of their physical characteristics. Bands 811, 812 and 813 and their corresponding graphical peaks in a graph 801 are of particular interest. Band 813 corresponds to a structural muscle protein (which might correspond to protein A in the discussion above). Band 811 corresponds to a breakdown product of the protein of band 813 (which might correspond to protein B in the discussion above). The appearance and quantitative amount of band 811 is therefore indicative of the degree of post-mortem proteolysis in the meat sample. Band 812 represents a protein whose quantity changes little in the post-mortem period. By looking at the corresponding peaks in the graph 801 for band 811 and 812, an accurate estimate of the degree of proteolysis can be obtained.

### Separation by Immunological Characteristics

In a second case, the degradation product can be detected through use of a lateral flow test, in either a competitive or a sandwich assay.

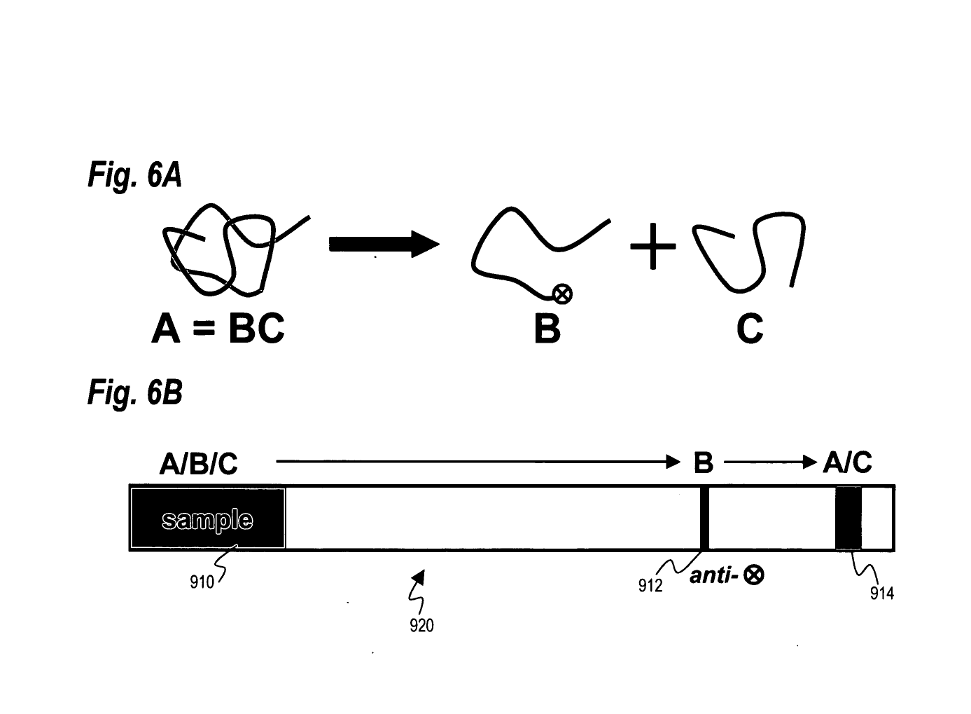

In a first case, an antibody, aptamer, or binding protein that binds to degradation product B, and not to protein A or degradation product C is available, and will be referred to as SBA (for Selective Binding Agent). FIG. 6A is a schematic diagram of a protein A that is degraded in situ by proteases to form degradation products B and C, in which product B has a Selective Binding Agent. The binding moiety is indicated in the diagram as a circle surrounding a cross, and can be located either at the location of the proteolysis (i.e. a terminus of B), or alternatively internal within B due to conformational changes in B that occur with proteolysis.

FIG. 6B is a schematic diagram of a lateral flow test 920 for detection of the protease degradation product B. In the lateral flow test 920, the SBA is placed in a fixed location on the test strip which will be referred to as the “SBA line” 912. It should be noted, however, that the actual shape of the SBA placement can be circular, a line, or any other shape with generally well defined boundaries. The SBA at line 912 binds to B as the degradation product B passes during the normal development of the lateral flow test 920. In a competitive assay, labeled B is competed with by B from the meat fluids, and in a sandwich assay, meat fluids are labeled with specific binding or non-specific binding colored, fluorescent, magnetic, enzymatic, catalytic or other labels.

It is possible to use labeled SBA and non-specific or specific binding at the SBA-line.

As can be seen, the captured B at line 912 gives indication of the degree of proteolysis within the meat sample. Undigested protein A and other degradation product C continue past the line 912, and accumulate at the “terminal” point 914, wherein this terminal point gives evidence of the total protein within the sample. The ratio of the quantity of B to the total protein is of preferable information to determine the amount of proteolysis. Alternatively, a second line with an antibody, aptamer or other SBA for A or some other protein present in muscle tissue can also be used instead of total protein content, such that two lines of reaction would be identified.

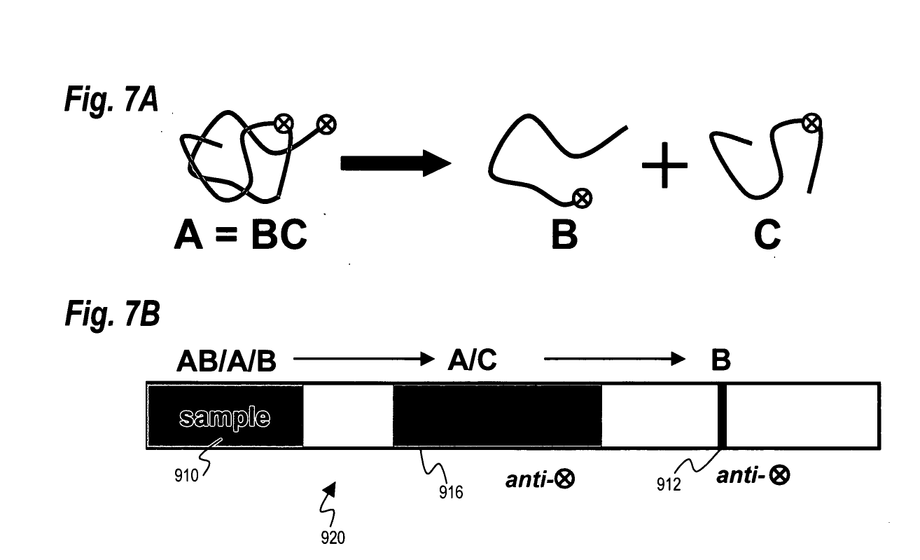

In another preferred embodiment, SBAs are prepared for both B and C (SBA-B and SBA-C), in which SBA-B has negligible affinity for C and SBA-C has negligible affinity for B. FIG. 7A is a schematic diagram of a protein A that is degraded in situ by proteases to form degradation products B and C, in which products B and C have Selective Binding Agents. Furthermore, SBA-C has significant affinity for the undegraded protein A. The binding moiety for SBA-B is indicated in the diagram as a black circle surrounding a black cross, whereas the binding moiety for SBA-C is indicated in the diagram as a gray circle surrounding a gray cross.

FIG. 7B is a schematic diagram of a lateral flow test 920 for detection of the protease degradation product B. In this case, in the direction of fluid travel from the spot of meat fluid application, there is first an SBA-C region 916, followed by an SBA-B line 912. The SBA-C region 916 absorbs all A and C, such that only protein degradation product B is detected at the SBA-B line 912. Indeed, detection of bound A and C at the SBA-C line 916 can serve as the control for the amount of B.

It should be noted that in this embodiment, not only can SBA-C have affinity for A, but also SBA-B can have affinity for A (since all of the A will have been “removed” by SBA-C before encountering the SBA-B line). Indeed, if the SBA-C line is being used as a control, then an affinity label with affinity for A can be used to indicate intensity at both SBA-B and SBA-C lines.

The intensity of the label collected at the SBA-B line 912 and, in the latter embodiment, the SBA-C line 916, is read by an instrument, which may be an imaging camera with incident visible or fluorescent illumination, or some other modality that is matched to the label used.

It can be convenient for there to be a control protein or compound unrelated to protein A that is used to normalize, for instance, for the amount of meat fluids that are applied to the strip. This would generally involve a selective binding agent that is specific for a compound in meat whose quantity is roughly unchanged during general proteolysis, and can include antibodies that recognize both the intact and the degraded versions of a specific protein. The amount of B and C can then be computed as a ratio relative to the control protein. The position of the SBA-line and the line for the control will be non-overlapping, but generally close.

SBAs for B or C that do not have affinity for A can be obtained in a number of different ways. In a first way, antibodies, aptamers or binding proteins can be raised against B, and then are washed on a column with A, thereby removing antibodies with an affinity for A. In a related manner, phage display libraries are prepared from animals challenged with B, the phage are washed on a column with A, and then bound with a column with B to find phage with the proper affinity. Those phage that remain have affinity for B but not A can be propagated to make homogeneous quantities of the phage with specific affinity for B.

The methods above assume that antibodies specific to a particular protein A and its degradation product B and C will be analyzed, but it is also possible to use antibodies with more general affinity for products that are present in degraded versus undegraded tissue. For example, proteins from aged meat can be used to generate antibodies, and these antibodies can be washed over a column with proteins from freshly slaughtered animals, thereby enriching for antibodies against degraded proteins.

It should also be appreciated that a number of different SBAs can be used in the test, with each SBA being placed onto the lateral flow strip in a different location. It is preferable for these different SBAs to be specific for different proteases, such as one SBA specific for a calpain substrate, and another specific for a caspase substrate.

In order to provide greater dynamic range for the lateral flow test, the SBA-line can be made wide with respect to the direction of flow, so that as part of the line becomes saturated, binding occurs over a wider area. Thus, instead of simply the intensity of the line, the width of the line above a given intensity is also diagnostic of the amount of B or C. Alternatively, the summed intensity over the width of the line can be used.

In the description of the lateral flow tests above, the placement of the capture binding protein is emphasized, and the descriptions do not show many of the other required aspects of a lateral flow assay, such as the sample pad, the conjugate pad with the detection conjugate, the terminal absorbent pad, the backing card, etc. Most of these aspects of a lateral flow strip are well-known in the art, and can be adjusted to achieve convenient processes for obtaining samples of intracellular fluids, developing the strip, or measuring the distribution of proteins (including automation of said processes). The number of possible arrangements within the present invention are extremely numerous. It should be appreciated that a useful aspect of the present invention is the preparation of Specific Binding Agents specific to at least one protein degradation product (B and/or C in the terminology above) and which show little binding with the undegraded protein from which they are derived (A in the terminology above).

Of interest to this discussion of the present invention are the characteristics of the detection conjugate. The indicator can comprise gold or silver, carbon, a visible or florescent dye, magnetic particles, enzymes, latex beads impregnated with visual or fluorescent dyes or other such indicators. The detection (that is, the attachment of the indicator to the proteins A, B or C is carried out by antibodies or aptamers that bind to these proteins. In the discussion above, the specificity of the assay is incorporated into SBAs which are affixed to the support, and the detection indicators can have broad reactivity with the proteins.

As an alternative, if the specific binding agent is not a protein, but, for example, an aptamer, then a general protein stain can be used to detect any protein located at the, for example, SBA-B line 912. This removes the need for a detection conjugate.

Alternatively, the specificity of the assay can be incorporated into the detection conjugates, while the antibodies, aptamers or other reagents affixed to the strip 920 have lesser specificity. That is, if only product B is labeled with indicator, a generalized binding reagent on the strip 920 would provide color only if product B were present.

Furthermore, both the detection conjugate and the affixed reagent can have matching specificity. For example, if the antibody to product B in the detection conjugate is a rabbit antibody, and the antibody to product C in the detection conjugate is a sheep antibody, then mouse antibodies to rabbit antibodies and sheep antibodies can be affixed onto the strip 920 at discrete locations in order to provide separate indications of the quantities of product B and product C present.

### Instrumentation

Separation based either on physical characteristics or on the basis of immunological characteristics can be performed in a variety of methods. Two commercially-viable methods and systems are described below. In a first method and system comprising column chromatography, small columns of a separation medium (e.g. adsorption chromatography or ion exchange resin) are prepared in small diameter (e.g. 1 mm) and inexpensive (glass tube) columns. A small amount of intracellular fluids obtained from the meat surface is deposited on the top of the column, and then the column is eluted via automated means, which can include spectrographic measurement of eluents.

In a second method and system using paper or thin layer chromatography, samples of intracellular fluid are obtained, placed onto the solid chromatographic support, and developed. The proteins and protein fragments are then located using stains that do not require destaining, such as that described in U.S. Pat. No. 6,316,267 to Bhalgat, et al, U.S. Pat. No. 5,616,502 to Haugland et al, or other stains known in the art.

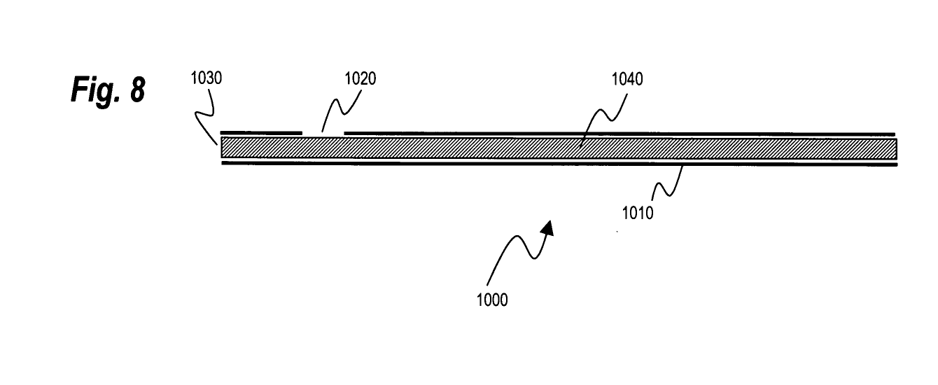

One method of obtaining samples and performing chromatography that have application in the present invention would be to place a thin-layer or paper chromatography test directly on a meat sample, in which only a spot or line of the substrate is uncovered and accessible to the meat sample. FIG. 8 is a side schematic view of a chromatographic separation strip 1000. The strip 1000 is coated on both sides by a water-impermeable coating 1010, which is transparent to the light used in the optical measurement of stained proteins at the conclusion of the chromatography. This coating can be permanent, or it can alternatively be removable at the conclusion of the chromatography to allow either unimpeded measurement of the protein, or to allow post-staining to occur.

The chromatography support 1040 can be a paper, and ion-exchange resin, silica gel, alumina or other such solid phase support. It should be appreciated that the impermeable coating 1010 on either side of the support 1040 can be different. For example, in the case of alumina as the support 1040, on one side, the coating 1010 can be glass or metal or stiff plastic, while on the other side, it can be a transparent film. In this way, the coating 1010 on one side can provide structural rigidity to prevent bending and cracking of the chromatographic support 1040.

A gap 1020 is provided on one surface of the strip 1000. When the side of the strip 1000 with the gap 1020 is placed onto a meat surface, intracellular fluid on or near the meat surface is wicked into the support 1040. In this way, a thin line or a circular dot of intracellular fluid is saturated on the support.

When the end of the strip 1000 is placed in a reservoir, the chromatography transport fluid is wicked at the end and moves through the strip 1000, thereby performing the chromatography. In order to reduce the number of handling steps, it is preferable for the chromatography transport fluid to incorporate the protein stain.

In the case of a lateral flow strip, a similar arrangement is possible, so that the sample pad is accessible and can be placed on the meat surface, whereas other parts of the lateral flow strip are inaccessible.

It should also be appreciated that the presence and quantities of different proteins B and C can also be determined by other methods than the chromatographic or electrophoretic or other separation techniques, but rather by standard methods within the analytic repertoire, such as ELISA methods or mass spectroscopy.

For example, with regards to mass spectroscopy, the hydrolysis at a specific target sequence of a prevalent structural protein can be detected distinctly from that of enzymes used, for example, for digesting the sample prior to mass spectroscopy. In such an embodiment, the small samples of meat, which can include intracellular fluid samples, are digested with trypsin, for example, which has a different target sequence from calpain, caspase or other proteases present in post-mortem meat samples. The digested samples are then analyzed by mass spectroscopy to determine the prevalence of fragments related to the intrinsic proteases within the meat sample.

An alternative to this methodology is the use of Laser Desorption Mass Spectrometry (LDMS), Matrix-Assisted Laser Desorption/Ionization (MALDI), and similar techniques. In such a case, a solid support or a matrix is used to collect the intracellular fluid from the surface of a meat sample. The matrix is then brought to the mass spectrogram, which laser desorption occurs and the mass spectrogram is run. If the protein breakdown product is of a reasonably prevalent source, such as collagen, actin, desmin, or other structural protein, the laser desorption and ionization can provide fragments of a size suitable for mass spectroscopic determination of the prevalence of protein breakdown products.

### Determination of Meat Tenderness

The methods and systems of the present invention can provide indications of the proteolysis of multiple proteins by a single protease, the proteolysis of multiple proteins acted on by multiple proteases, measures of calcium levels or pH in addition to protease activity, and in simple extension of the principles given herein, to the measurement of the degradation of carbohydrates, DNA, RNA, and other metabolic events post-mortem. Furthermore, by tracking multiple proteases (through the measurement of multiple protein breakdown products), a clearer view of the nature of the meat sample tissue can be obtained. For example, calpains appear to be more associated with necrosis, whereas caspases appear to be more associated with apoptosis, which should result in a more complete proteolysis of the meat sample. By looking at the differences in the quantities of protein breakdown products results from calpain proteolysis and the quantities of protein breakdown products results from caspase proteolysis, or through measurement of calpain versus caspase protease activity, a better sense of the nature of tissue death, which is associated with meat tenderness, can be ontained.

The aim of these techniques is not to measure protein degradation, pH, calcium levels and the like, but rather to measure meat tenderness. Each one of the points of information can be considered to be a feature of the meat sample. Such features are put into a decision algorithm so as to determine meat tenderness.

There are a variety of different decision algorithms that can be employed that use the features described above as input, and which output either a continuous tenderness measurement, or alternatively a discrete classification such as tender or tough. This latter classification type will generally correspond to a specific shear force threshold, such as the use of 19, 20, or 21 kg of sliced shear force as the threshold between tough and tender.

Conventional linear regressions, logistic regressions, Bayesian analysis, neural networks, support vector machines, and other methods currently in use as decision algorithms should be considered. Two methods, however, are of particular convenience. Before discussing these two methods, it should be noted that there are two distinct stages in the decision algorithm analysis. In a first stage, the features from a set of samples for which actual tenderness is been measured using conventional methodology such as sliced shear force or the Warner-Bratzler test are used to determine decision algorithm parameters. In the second stage, the decision algorithm parameters are used in conjunction with features measured from samples of unknown tenderness so as to compute a predicted tenderness. The discussion below primarily deals with the generation of the decision algorithm parameters in the first stage, as the second stage is a straightforward application of the decision algorithm well known in the prior art.

In the first method, the features can be used as inputs to random forests. The output to the random forest is the measured tenderness, which can be either a continuous pressure or conveniently a tough/tender classification. The use of random forests is generally useful in this application due to the large number of features that can be generated and the ability of random forests to handle large numbers of features.

In the second method, feature values are used as discrete thresholds in a binning procedure. For example, given two features A and B, a threshold for feature A and a threshold for feature B are selected such that they provide the greatest meat tenderness discrimination. Such a binning procedure can be extended to as many features as are necessary in order to provide adequate meat tenderness discrimination.

It should be noted that the objective function for tenderness discrimination can be of a variety of different formats. In a first format, the objective function can be the sum for all bins of the absolute value of the difference between the fraction of the tough meat in the bin and the average fraction of tough meat in the entire sample.

In a second format, the objective function can be the fraction of all the meat in bins that exceed a predetermined fraction that are tough or tender. Because tender meat that is in a tough bin is downgraded in value, tough bins that are only 40 to 60% tough are of considerably lower value than tough bins that are 70% to 100% tough. Therefore, it is preferable for tough bins to have greater than 60% tough meat, and more preferably greater than 70% tough meat, and most preferably greater than 75% tough meat. Likewise, in order to guarantee tenderness to consumers, a bin can be in general preferably greater than 90%, and more preferably greater than 95%, and most preferably greater than 97% tender.

It should be understood that a meat processing facility that incorporates meat tenderness determination already has a representative fraction of tough and tender meat. Such a facility already has marketing channels that allow it to sell meat of the default representative fraction. In a third format, three bins instead of two bins can be conveniently used. The first set of bins will be for tender meat, the second set of bins will be for tough meat, and the third set of bins will be for meat of the representative fraction. This is best illustrated through an example. In the tender bin, meat with a 95% or greater tender fraction is collected. In the tough bin, meat with the 60% or greater tough fraction is collected. In the default bin, meat with a 15 to 25% tough fraction is collected (assuming that the default representative fraction is 15 to 25%). Bins with 5 to 15% tough fraction contain tender meat in them to do not adequately reward higher fractions of tender meat (e.g. they do not meet the 95% threshold). On the other hand, bins with 25 to 60% tough fraction downgrade a very large amount of tender meat that is contained along with the tough meat.

One way of considering this third format is to enumerate objective goals. The first goal is to have tender bins in which tender meat exceeds a predetermined fraction of the meat. The second goal is to make the tender bins as large as possible. The third goal is to have tough bins in which tough meat exceeds a predetermined fraction of the meat. The fourth goal is to have a default bin in which the fraction of tough meat is preferably within 3% of the default representative fraction, and more preferably within 5% of the default representative fraction, and most preferably within 8% of the default representative fraction. The fifth goal is to reduce as much as possible the amount of meat in the default bin.

It should be noted that with four features, there will generally be 16 bins. In the previous paragraphs discussing alternative formats, it should be noted that the bins will generally be the sum of all of the bins needing a particular criterion. For example, if there are three bins where the fraction of tender meat exceeds the predetermined threshold, the tender bins will be the sum of all three bins.

It should be appreciated that values or attributes used in the decision algorithm need not be obtained through the use of high resolution imaging. For example, other information that is obtained that has potential value in an algorithm might include ribeye size, gender, age, marbling score, or other information. The features of the present invention are not necessarily meant to substitute completely for attributes or values obtained by other means. To the extent that such information improves the tenderness determination, it should be included in the decision algorithm.

### Other Biomolecules

It should be appreciated that while the focus of the present invention is proteins, other biomolecules within an animal undergo degradation during the post-mortem period. In particular, these include nucleic acids, lipids and carbohydrates. The breakdown of these biomolecules can indicate either changes in physiology within the muscle tissue (e.g. the exhaustion of carbohydrate stores), or the degree or manner of cell death within the tissue (e.g. cells in stasis, cells dying by necrosis, cells dying by apoptosis).

The methods of the present invention can apply equally to the other biomolecules as they do to proteins. For example, the presence of low molecular weight DNA (corresponding to protein breakdown products, but with respect to DNA instead of proteins) signal the occurrence of apoptosis, whereas medium molecular weight DNA would be more indicative of necrosis.

### SUMMARY OF TERMS

This Summary of Terms provides a convenient condensation of terminology used in this specification, which should not be limiting and should be considered in combination with further explication elsewhere in this specification, or as used or understood by those skilled in the art.

Meat sample comprises a carcass, or a piece of a carcass (e.g. a loin, a steak, or ground up meat) that comes from a single animal for which a tenderness measurement is desired. In commercial applications, a rib-eye surface will be exposed on the grading line, and this is the meat sample that will frequently be used in the determination. However, it is also possible that such a sample will come from a large-gauge needle biopsy of a live animal. It should be noted that many different measurements can be obtained for a single meat sample (e.g. from different places on a carcass), so as to provide a better single measurement of meat tenderness.

Test strip comprises a solid matrix and other components on which a measurement of protease activity or protein degradation product quantity is performed. The solid matrix can comprise a piece of porous, flexible material made with natural materials (e.g. cellulose) or artificial materials (e.g. nitrocellulose or an ion-exchange paper), or it can comprise a non-porous material (e.g. a flexible piece of plastic, or a glass slide) on which a powder or other material (e.g. silica or ion-exchange resin beads) is attached. This test strip can either be applied directly to the meat sample, or a fluid from the meat sample can be collected, which is then applied to the test strip. It should be noted that the test strip can comprise a measurement for a single meat sample, or it can also be that measurements for many different meat samples can be performed on a single test strip.

Protease refers to an enzyme that hydrolyzes peptide bonds in a protein. In tests of meat tenderness, this will often comprise a calpain, a caspase or a cathepsin. However, in many cases of the present invention, the specific protease causing a specific hydrolysis event will not be identified, and it can comprise proteases other than those mentioned above.

Protease substrate comprises a polypeptide sample that is cleaved by a protease. This polypeptide sample can comprise a single natural protein (e.g. actin, myosin, desmin, collagen, or other protein obtained directly from a protein sample, or purified from a natural protein sample), or a modified natural protein (e.g. labeled with an optically-detectable marker, partially hydrolyzed to make it soluble, covalently bonded to a solid support). The polypeptide sample can comprise a single synthetic polypeptide, which can comprise a polymer of one or more synthetically-generated protease recognition sequences. If there are a multiplicity of synthetically-generated protease recognition sequences, the sequences within a polypeptide can be a repeated single recognition sequence specific for a single protease, multiple recognition sequences specific for a single protease, or can include multiple recognition sequences for different proteases. The polypeptide sample can comprise a combination of many different natural proteins, modified natural proteins and/or synthetic polypeptides, such as hydrolyzed muscle protein, or a sample of intracellular fluid collected from the surface of a meat sample.

A marker comprises something attached to an object of interest, such as a protease substrate, that allows the object to be tracked. In general, this marker is an optically-detectable marker, which means that an optical means can detect its presence, and in general its location and its quantity. In the present invention, a marker often comprises a chromophore or a fluorophore.

Discrete location comprises a location that has well defined boundaries. For example, placing a spot of labeled protease substrate on a test strip at a discrete location means that a specific spot on the test strip either has labeled protease substrate or it does not, and that places do not have intermediate levels of protease substrate. It should be appreciated that in practice, such discrete boundaries cannot be absolute, and that the meaning of the term depends in part on the context of the test being performed.

An imager comprises a device that captures a two-dimensional image coincidentally.

A scanner comprises a device that captures a one-dimensional profile or a two-dimensional image over a period of time.

Optical profiler comprises a means to measure the location and quantity of an optically-detectable marker on a test strip. The optical profiler can detect either the one or two-dimensional distribution of the marker, and depending on the marker, can detect in visible, infrared or ultraviolet wavelengths, and/or can comprise a detector capable of exciting fluorescence markers and detecting fluorescence emissions, or of detecting luminescence. The optical profiler can operate on principals of absorbance or transmittance. The optical profile can comprise an imager (e.g. a CCD or CMOS camera), or it can be a laser imager, or a laser or CCD scanner, or other such device comprising a light detector. The optical profiler can comprise a black and white detector, a single wavelength detector, a color camera, or a spectrophotometer. It should be noted that an alternative arrangement of the device is to have a throughput separation of proteins, such as through a micro-ion-exchange column, in which proteins pass through or are eluted at different times. In such case, a profiler takes continuous optical readings of the device, providing a one-dimensional profile of the output of the throughput device.

Protein breakdown product comprises the product of a proteolysis of a protease substrate, which can also be referred to as a source. For example, if the source is actin, the protein breakdown product can be a lower molecular weight fragment produced by the action of a protease on the actin. In the context of a protein sample from a natural source (e.g. a beef carcass 24-hours post-mortem), the source is typically a natural muscle protein, and the protein breakdown product is the result of proteolytic activity intrinsic to the muscle.

Protein breakdown complement comprises the complementary protein fragment to a protein breakdown product. For example, if the source is actin, and it is hydrolyzed by a protease, the hydrolysis event produces two fragments. One fragment comprises the protein breakdown product, and the other is a protein breakdown complement. The definition of one fragment as product and the other as complement is symmetrical and contextual, and in other contexts, what is the product can be the complement and visa versa. It should also be understood that for a source protein and a protein breakdown product, there can be many complements. In general, given a protein breakdown product, there will be no amino acid overlap between the product and all of the related complements.

Reference comprises a material that is used as a quantity reference in the measurement of some other material. In the present invention, this often refers to a protein in a protein sample that is relatively constant from animal to animal. For example, the amount of protein sample collected from a meat sample onto a test strip can be varied, and the protein sample can also comprise different amounts of intracellular fluids, blood, ambient water spray, and more. When measuring a material such as a protein breakdown product, the quantity is most meaningful in relation to a reference. This reference could be a structural material such as actin, or a protein in roughly equal amounts in every cell, such as nuclear proteins (e.g. histones). The reference could also be in reference to total protein within the sample. It should be appreciated that in many cases, a source can be used as a reference for its corresponding protein breakdown product.

Sample collector comprises a means of collecting a protein sample from a meat sample. In the case of a test strip, the strip itself can function as the sample collector. For example, the test strip can be applied to the surface of the meat sample, absorbing intracellular fluids. Alternatively, a fluid collector can comprise one or more small diameter glass tubes that take up intracellular fluids via capillary action. Also, a fluid collector can comprise a device that cuts off a small piece of muscle tissue from the meat sample, grinds it, and then expresses a small amount of fluid under pressure.

Protein quantifier comprises a means for determining the quantity of a protein. The protein quantifier can comprise an optical profiler if the protein is either labeled by an optically-detectable marker, if the protein is stained with an optically-detectable stain, or if the protein has optical characteristics that can be observed (e.g. myoglobin is optically-detectable due to the iron-porphyrin, or due to the natural IR absorbance of most proteins). It should be appreciated that the methods of the present invention usually describe the use of optical means, but that other means of quantification are available. For example, as mentioned above, the use of mass spectroscopy also comprises a protein quantifier.

A separation medium comprises a material that can separate two polypeptides or proteins on the basis of their physical characteristics. The separation medium can comprise cellulose paper, nitrocellulose paper, ion-exchange paper or resin, cellulose, dextran gels, silica gel, polyacrylamide, agarose, or many of other media known in the art. The medium can be either in strip form, laid out on a solid substrate, packed into a column, or other such arrangement. In general, the two proteins are mixed together into a sample, and are placed at a single location on the medium. Then a separation impetus acts to on the proteins within the context of the medium to separate the proteins. Examples of separation impetuses include a salt solution, an organic solvent, or, in the case of an electrophoresis separation medium, an electrical current. It should be appreciated that various binding agents, such as antibodies or aptamers, can also be used in such a format to provide either discrete separation (e.g. protein A binds at location A where binding agent for A is located, and protein B binds at location B where binding agent for B is located), or in the case where there is a binding agent X uniformly distributed, with low binding and a small binding differential between proteins A and B, a gradual separation over a distance between the two proteins. It should be noted that separation can be in time or place. In the case of a test strip, proteins are separated into two different locations, which are then measured. In the case of a throughput device, such as an ion-exchange column, the proteins are separated by differential time movement through the device, and pass through the device at different times.

Physical characteristics used in separation comprise in general a combination of a number of characteristics, which can comprise protein pIs, size, aspect ratio, hydrophobicity, hydrophilicity, secondary, tertiary and quaternary structures, and many other factors.

Binding reagents comprise reagents that are specific for a targeted protein. Examples of such binding reagents include antibodies, antibody fragments, aptamers, lectin, a membrane receptor or other such reagent. It should be appreciated that such binding is never absolute either in its degree or its specificity, and that relative binding strength and specificity are of practical usefulness in the present invention.

An affinity linker comprises a binding reagent that is associated with a marker, which is used to mark a protein. For example, a labeled mouse antibody against a cow muscle protein comprises an affinity linker, as it labels the cow muscle protein with the marker. In general, the affinity linker comprises a binding reagent to accomplish the linking. Usually, the linkage that occurs is not a covalent linkage.

Immobilizers comprise a binding reagent that is used to immobilize a protein to a specific location. For example, a rabbit anti-mouse antibody can immobilize a cow muscle protein which has been linked to a labeled mouse anti-cow muscle protein antibody affinity linker. Another example would be an aptamer that binds tightly to actin, wherein the aptamer is bound to a lateral flow strip at a discrete location and binds actin which has been labeled.

The starting location, labeling location, and binding location comprise regions along a test strip at which different activities occur. The protein sample is generally deposited at the starting location, which is conveniently a spot or a line on the test strip. The labeling location is where one or more proteins are labeled with markers, usually with some form of affinity linker. The binding location is where the protein, which can at this point in the process have been labeled with an affinity linker, or which can be intrinsically labeled, or which can be unlabeled at this point, is immobilized by an immobilizer.

Distinguishable locations comprise two locations at which two different proteins are being quantified, wherein the amount being quantified at one location is roughly independent of the amount that is quantified at the other location. For example, if a chromatographic separation gives two bands, the ability to quantify the amounts of material in the two bands depends on the bands being distinguishable. If the bands are substantially overlapping, some degree of quantitation is possible, but more difficult, especially if one band is of much greater intensity than the other band, if there is substantial noise present, if the width of the bands is small relative to the resolution of the measuring instrumentation, or other factors.

Decision algorithms comprise any mapping or other process of assigning features from the high-resolution imaging, and which can additionally include values or attributes determined by means other than high-resolution imaging, into a meat tenderness determination.

Meat tenderness determination comprises the derivation of both qualitative and quantitative measures. In one sense, meat tenderness can comprise a value that can be compared with a predetermined value, such that being on one side or another of the predetermined value indicates the tenderness of the meat. In another sense, meat tenderness can comprise a set of categories, such as “tender”, “tough” and “intermediate” that is assigned to the meat. In yet another sense, meat tenderness can comprise a probability of the meat being a member of a category. It should be noted that the measurement of tenderness is typically done in the first 48 hours post-mortem, whereas the consumption of meat occurs typically 14-28 days post-mortem. Therefore, the meat tenderness measurement can actually be a prediction of the meat tenderness that will occur at a later time period. From the processes of commercial meat packing plants, it is preferable for the time at which the analysis be performed be between 18 and 48 hours post-mortem.

Structural protein comprises an abundant protein that is generally considered to be part of the structural elements of a cell, and can comprise a form of actin, myosin, desmin, collagen, tubulin, etc.

### Many Embodiments Within the Spirit of the Present Invention

It should be apparent to one skilled in the art that the above-mentioned embodiments are merely illustrations of a few of the many possible specific embodiments of the present invention. It should also be appreciated that the methods of the present invention provide a nearly uncountable number of arrangements.

Numerous and varied other arrangements can be readily devised by those skilled in the art without departing from the spirit and scope of the invention. Moreover, all statements herein reciting principles, aspects and embodiments of the present invention, as well as specific examples thereof, are intended to encompass both structural and functional equivalents thereof. Additionally, it is intended that such equivalents include both currently known equivalents as well as equivalents developed in the future, i.e. any elements developed that perform the same function, regardless of structure.

In the specification hereof any element expressed as a means for performing a specified function is intended to encompass any way of performing that function. The invention as defined by such specification resides in the fact that the functionalities provided by the various recited means are combined and brought together in the manner which the specification calls for. Applicant thus regards any means which can provide those functionalities as equivalent as those shown herein.

What is claimed is:

1. A method for determining tenderness of a meat sample, comprising:
placing a test strip on the meat sample, wherein the test strip comprises a solid support and a protease substrate bound at a discrete location on the support, wherein the protease substrate is labeled with an optically-detectable marker, and wherein the protease substrate and the marker are inhibited from diffusing from the discrete location;
putting the test strip into contact with fluids from the meat sample, so that the fluids bathe the labeled protease substrate;
incubating the test strip for a predetermined period of time, in which proteolysis hydrolyzes the protein substrate and releases the marker from the discrete location;
measuring the diffusion of the optically-detectable marker from the discrete location; and
determining tenderness of the meat sample using an automated decision algorithm that uses the amount of diffusion of the marker.

2. The method of claim 1, wherein the protease substrate comprises a structural protein.

3. The method of claim 1, wherein the protease substrate comprises a polypeptide comprising multiple protease recognition sites.

4. (canceled)

5. The method of claim 1, wherein the predetermined period of time is less than 20 minutes.

6. (canceled)

7. The method of claim 1, wherein the measuring means comprises an imager.

8. The method of claim 1, wherein the measuring means comprises a scanner.

9. The method of claim 1, wherein the step of measuring occurs while the test strip is in contact with the meat sample.

10. The method of claim 1, wherein the step of measuring occurs while the test strip is not in contact with the meat sample.

11. The method of claim 1, wherein the step of measuring further comprises measuring the optically-detectable marker at the discrete location.

12. The method of claim 1, further comprising obtaining an indication of the pH of the meat sample from an indicator on the test strip.

13. The method of claim 1, further comprising obtaining an indication of the concentration of calcium ions within the meat sample from an indicator on the test strip.

14.-18. (canceled)

19. A method for determining tenderness of a meat sample, comprising:
obtaining a protein sample from the meat sample, wherein the protein sample comprises a protein breakdown product resulting from post-mortem proteolysis and a reference;
separating the protein breakdown product from the reference on the basis of their physical characteristics;
measuring the quantity of the protein breakdown product relative to the reference; and
determining tenderness of the meat sample using an automated decisional algorithm that uses the measured relative quantity.

20.-34. (canceled)

35. The method of claim 19, wherein the separating comprises ion-exchange.

36. The method of claim 19, wherein the separating comprises chromatography.

37. The method of claim 19, wherein the separating comprises mass spectroscopy.

38. The method of claim 37, wherein the mass spectroscopy is preceded by laser desorption.

39. The method of claim 19, wherein the separating comprises electrophoresis.

40. The method of claim 19, wherein the step of obtaining further comprises contacting the surface of the meat sample with a separation medium.

41. (canceled)

42. A method for determining tenderness of a meat sample, comprising: obtaining a protein sample from the meat sample, wherein the protein samples comprises a protein breakdown product resulting from post-mortem proteolysis of a source protein, a protein breakdown complement that is formed coincidentally to the protein breakdown product during formation by proteolysis of the source protein, and a reference; separating the protein breakdown product from the source protein and the protein breakdown complement using a binding reagent that binds to the protein breakdown product and which does not bind to the source protein, the protein breakdown complement or the reference; measuring the quantity of the protein breakdown product relative to the reference; and determining tenderness of the meat sample using an automated decision algorithm that uses the measured relative quantity.

43. The method of claim 42, wherein the binding reagent is selected from the group consisting of antibodies, antibody fragments, aptamers, lectins and membrane receptors.

44. The method of claim 42, wherein the step of separating further comprises attaching the binding reagent to a location on a solid support, and collecting the protein breakdown product at that location.

45. The method of claim 44, wherein the step of separating comprises binding a second binding reagent to the reference, which is attached to the solid support at a second location on the solid support.

46. The method of claim 45, wherein during the separating, the protein breakdown product and the reference contact first the first location.

47. The method of claim 45, wherein during the separating, the protein breakdown product and the reference contact first the second location.

48. The method of claim 42, wherein the reference comprises the source protein.

49. (canceled)

50. A method for determining tenderness of a meat sample, comprising:
transferring a protein sample from the meat sample onto a starting location on a test strip; transporting the protein sample from the starting location to a labeling location; labeling a protein breakdown product within the protein sample with an optically-detectable marker at the labeling location; conveying the labeled protein breakdown product to a binding location; binding the labeled protein breakdown product to the binding location; profiling the optically-detectable marker at the binding location in order to determine the quantity of protein breakdown product in the protein sample; and determining the tenderness of the meat sample from the quantity of the protein breakdown product.

51. The method of claim 50, wherein the step of labeling further comprises binding to the protein breakdown product a labeled binding agent that is specific for the protein breakdown product and that is stored at the labeling location to the protein breakdown product.

52. The method of claim 50, wherein binding comprises binding the labeled protein breakdown product to a binding agent that is specific for the protein breakdown product and that is attached to the test strip at the binding location.

53. The method of claim 50, wherein the test strip comprises a lateral flow test strip.

54. A method for determining tenderness of a meat sample, comprising:
transferring a protein sample from the meat sample onto a starting location on a test strip, wherein the protein sample comprises a protein breakdown product and a reference;
conveying the protein breakdown product and the reference along the test strip, wherein the movement of the protein breakdown product and the reference are at different rates;
staining the test strip so as to make the protein breakdown product and the reference optically-detectable;
profiling the test strip with an optical profiler so as to measure the quantities of protein breakdown product and the reference, which are at distinguishable locations on the test strip; and
determining the tenderness of the meat from the quantities profiled by the optical profiler.

55. The method of claim 54, wherein the test strip comprises a chromatography strip.

56. The method of claim 54, wherein the step of transferring further comprises contacting the test strip to the meat sample.

57. The method of claim 56, in which the surface of the test strip contacting the meat sample is coated with a non-porous coating comprising a gap, wherein transfer of the protein sample to the test strip is limited to the location of the gap.
